(19) United States
(12) Patent Application Publication
(10) Pub. No.: US 2018/0099016 A1
(43) Pub. Date: Apr. 12, 2018

(54) USE OF AFRICAN SEED EXTRACT FOR HUNGER SUPPRESSION

(71) Applicant: ENGEN MEDICAL CORPORATION, San Diego, CA (US)

(72) Inventors: PAUL EPNER, SAN DIEGO, CA (US); RICHARD ZIMMER, III, MANDEVILLE, LA (US); BILL W. MASSEY, HEBER SPRINGS, AR (US)

(73) Assignee: ENGEN MEDICAL CORPORATION

(21) Appl. No.: 15/728,657

(22) Filed: Oct. 10, 2017

Related U.S. Application Data
(60) Provisional application No. 62/406,202, filed on Oct. 10, 2016.

(51) Int. Cl. A61K 36/185; A61K 9/68; A61K 9/20

(52) U.S. Cl. CPC A61K 36/185; A61K 9/0058; A61K 9/2013; A61K 9/2018; A61K 9/2009

(57) ABSTRACT

A composition and method for the use of Irvingia gabonensis seed extract for hunger suppression in obese individuals with low Leptin sensitivity, individuals with higher levels of ghrelin, and for individuals with Cannabis-induced appetite stimulation where dosage can be effectively administered in a gum base cold pressed into a tablet containing at least 100 mg of Irvingia gabonensis seed extract and Maltitol, Sorbitol, Isomalt, Xylitol, natural & artificial flavors, vegetarian magnesium stearate, Sucralose and Silicon dioxide.

This application claims priority from U.S. Provisional Application No. 62/406202 (the '202 application), filed Oct. 10, 2016. The '202 application is incorporated herein by reference.

### BACKGROUND OF THE INVENTION

Leptin (from Greek λεπτóç leptos, “thin”), also known as the satiety hormone, is a hormone made by adipose cells that helps to regulate energy balance by inhibiting hunger. Leptin has been shown to act on receptors in the arcuate nucleus of the hypothalamus to regulate appetite to achieve energy homeostasis. Following eating, leptin levels increase and biological sequelae resultant from leptin interacting with the leptin receptor produce the feeling of satiety and decrease appetite. In obesity, a decreased sensitivity to leptin occurs, either from eating-induced resetting of appetite or from dysfunctional genetic mutations of the leptin receptor, resulting in an inability to detect satiety despite high energy stores.

Cannabis (aka marijuana) is a genus of flowering plants that consists of three subspecies, Cannabis sativa, Cannabis indica, and Cannabis ruderalis. Cannabis produces hundreds of cannabinoids, which are terpeno-phenolic compounds. Some of these cannabinoids produce psychoactive effects (e.g. delta-9-tertrahydrocannabinol) whereas other cannabinoids are biologically-active and produce wide-ranging effects as disparate as conjunctive vasodilation, immune system regulation, stimulation of appetite, sleep-induction, and analgesia. Medical use of Cannabis is practiced today and has a history going back thousands of years, however, the medical utility of Cannabis is a topic of much contention. Several states have legalized the recreational and medical use of Cannabis and more states have placed Cannabis legalization on ballots for the voters of these states to determine its legal status. Currently, the US Federal government does not recognize the medical utility of Cannabis but has not interceded in the sale and distribution of Cannabis in those states where its use has been legalized. The eventual legal status of Cannabis at the federal level is uncertain. Despite the confusing and evolving legal status of Cannabis, it is one of the most commonly-used psychoactive substances in the world, exceeded only by ethanol-containing beverages, tobacco (nicotine), and coffee (caffeine). One of the known biological effects of the consumption of cannabis is the stimulation of appetite. This effect is commonly known as the “munchies” and results in increased consumption of food following Cannabis use. The mechanism for Cannabis-induced appetite stimulation is via agonism of cannabinoid subtype 1 (CB1) receptors. Agonism of CB1 receptors results in the increased release of ghrelin, a hormone that increases appetite, and inhibition of leptin release, both of which work in concert to increase appetite (aka hunger) and decrease food satiation. Thus, leptin and ghrelin, and the balance between their levels, are the primary hormones involved in regulation of appetite. Cannabis use, via interaction of cannabinoids with the CB1 receptor, results in increased ghrelin release and decreased leptin expression, resulting in increased hypothalamic-induced hunger and reduced satiety, respectively, that is manifested as increased appetite and desire to consume food. The stimulation of appetite is sometimes an unwanted effect of Cannabis consumption, and users may desire a means to control this unwanted effect.

### SUMMARY OF THE INVENTION

It is an object of this invention to provide a composition of Irvingia gabonensis seed extract comprising a dosage in an amount effective for hunger suppression in obese individuals with low Leptin sensitivity, individuals with higher levels of ghrelin, and for individuals with Cannabis-induced appetite stimulation.

It is a further object of this invention to provide the composition of Irvingia gabonensis seed extract within a gum base for efficacious delivery to the user through the ordinary process of gum chewing, where the gum base further comprises Maltitol, Sorbitol, Isomalt, Xylitol and other components.

It is a further object of this invention to provide a method for hunger suppression in obese individuals with low Leptin sensitivity, individuals with higher levels of ghrelin, and for individuals with Cannabis-induced appetite stimulation comprising the administering of a composition of Irvingia gabonensis seed extract in an effective amount and where that composition is within a gum base further comprising Maltitol, Sorbitol, Isomalt, Xylitol and other components for efficacious delivery to the user through the ordinary process of gum chewing.

### DETAILED DESCRIPTION OF THE INVENTION

The present invention described herein is the use of the seed of Irvingia gabonensis (aka African mango tree), including any processed (e.g. extracts, conjugates, heated, cooked, etc.) or unprocessed form of the seed, for those with low Leptin sensitivity as well as for the attenuation of Cannabis-induced appetite stimulation incorporated within a gum base with the below included ingredients for efficacious delivery to the user through the ordinary process of gum chewing. The gum base is formulated utilizing a cold pressed manufacturing process to incorporate all of the ingredients with heightened efficacy into a circular half inch diameter tablet. The ingredients incorporated into the gumbase are, Maltitol, Sorbitol, Isomalt, Xylitol, African mango seed extract (Irvingia gabonensis) 100 mg per piece, natural & artificial flavors, vegetarian magnesium stearate, Sucralose and Silicon dioxide.

Irvingia gabonensis is a fruit-producing tree native to the forested regions of central Africa, including the countries of Congo, Nigeria, Cameroon, Ghana, Togo, and Uganda. The seeds of Irvingia gabonensis are edible and are used for various purposes, including appetite suppression. The appetite suppressive effects of Irvingia gabonensis has been shown to result from increased leptin expression and/or enhanced leptin sensitivity (Oben J E, Ngondi J L, Blum K. Inhibition of Irvingia gabonensis seed extract (OB131) on adipogenesis as mediated via down regulation of the PPARgamma and leptin genes and up-regulation of the adiponectin gene. Lipids Health Dis. 7:44, 2008). These leptin-related effects and properties of the seeds of Irvingia gabonensis specifically counteract low leptin sensitivity and a mechanism of cannabis-induced appetite stimulation and as such is postulated to have utility in the attenuation of appetite stimulation. Initial subjective trials of Irvingia gabonensis seed extract have been conducted by the inventors in adult volunteer normal dieting and Cannabis users and self-reported to be effective in attenuating low leptin sensitivity and Cannabis-induced appetite stimulation.

What is claimed is:

1. A composition of Irvingia gabonensis seed extract comprising a dosage in an amount effective for hunger suppression in obese individuals with low Leptin sensitivity, individuals with higher levels of ghrelin and for individuals with Cannabis-induced appetite stimulation.

2. The composition of claim 1 where the composition of Irvingia gabonensis seed extract is incorporated within a gum base for efficacious delivery to the user through the ordinary process of gum chewing.

3. The composition of claim 2 where the gum base is cold pressed into a tablet containing Irvingia gabonensis seed extract in an amount of 100 mg.

4. The composition of claim 3 where the gum base further comprises Maltitol, Sorbitol, Isomalt, Xylitol, natural & artificial flavors, vegetarian magnesium stearate, Sucralose and Silicon dioxide.

5. A method for hunger suppression in obese individuals with low Leptin sensitivity, individuals with higher levels of ghrelin, and for individuals with Cannabis-induced appetite stimulation comprising the administering of an effective amount of a composition of Irvingia gabonensis seed extract where the amount of Irvingia gabonensis seed extract is 100 mg.

6. The method of claim 5 where the composition of Irvingia gabonensis seed extract is incorporated within a gum base for efficacious delivery to the user through the ordinary process of gum chewing.

7. The method of claim 6 where the gum base is cold pressed into a tablet further comprising Maltitol, Sorbitol, Isomalt, Xylitol, natural & artificial flavors, vegetarian magnesium stearate, Sucralose and Silicon dioxide.
